(19) United States
(12) Patent Application Publication
(10) Pub. No.: US 2025/0360277 A1
(43) Pub. Date: Nov. 27, 2025

(54) INHALER AND INHALING SYSTEM INCLUDING THE SAME

(71) Applicant: KT&G CORPORATION, Daejeon (KR)

(72) Inventors: Yongmi JUNG, Daejeon (KR); Moonwon KIM, Daejeon (KR); Jun Won SHIN, Daejeon (KR)

(73) Assignee: KT&G CORPORATION, Daejeon (KR)

(21) Appl. No.: 19/092,129

(22) Filed: Mar. 27, 2025

(30) Foreign Application Priority Data
May 22, 2024 (KR) 10-2024-0066207

(51) Int. Cl. A61M 15/00

(52) U.S. Cl. CPC A61M 15/002; A61M 15/0035

(57) ABSTRACT

An inhaler into which a functional material accommodating article accommodating a functional material is inserted includes a housing, a sliding structure accommodating in the housing and movable in a longitudinal direction, and a needle accommodated in the housing, wherein an air inlet communicating with the insertion space is formed in the housing, and air outside the housing moves through the air inlet, passes through the sliding structure, and reaches the functional material accommodating article.

### CROSS-REFERENCE TO RELATED APPLICATION

This application claims the benefit of Korean Patent Application No. 10-2024-0066207, filed on May 22, 2024, in the Korean Intellectual Property Office, the entire disclosure of which is incorporated by reference herein for all purposes.

### BACKGROUND

### 1. Field of the Invention

The following embodiments relate to an inhaler and an inhaling system including the inhaler.

### 2. Description of the Related Art

An inhaler may transfer a target material directly to the lungs of a user. For example, KR Patent Application Publication No. 2000-7010085 discloses a nicotine inhaler.

The above description is information the inventor(s) acquired during the course of conceiving the present disclosure, or already possessed at the time, and is not necessarily art publicly known before the present application was filed.

### SUMMARY

Embodiments provide an inhaler that may induce smooth discharge of a functional material and an inhaling system including the inhaler.

Embodiments provide an inhaler that may induce vortex behavior of a functional material and an inhaling system including the inhaler.

Embodiments provide an inhaler that may implement tangential inflow of airflow and an inhaling system including the inhaler.

Embodiments provide an inhaler that does not require complex manufacturing facilities because the inhaler does not require forming a tangential airflow hole in a functional material accommodating article and an inhaling system including the inhaler.

Embodiments provide an inhaler that may enhance aesthetics and an inhaling system including the inhaler.

The technical aspects obtainable from the present disclosure are non-limited by the above-mentioned technical aspects, and other unmentioned technical aspects can be clearly understood from the following description by those having ordinary skill in the technical field to which the present disclosure pertains.

According to an aspect, there is provided an inhaler into which a functional material accommodating article accommodating a functional material is inserted, the inhaler including a housing including a first surface, a second surface that is opposite to the first surface, and a side surface between the first surface and the second surface and including an insertion space into which the functional material accommodating article is inserted from the first surface, a sliding structure accommodated in the housing and movable in a longitudinal direction connecting the first surface to the second surface, and a needle accommodated in the housing and protruding from the second surface toward the first surface, wherein the functional material accommodating article may be inserted through the first surface of the housing, wherein the housing may include an air inlet communicating with the insertion space, and wherein air outside the housing may move through the air inlet, pass through the sliding structure, and reach the functional material accommodating article

According to an aspect, there is provided an inhaling system including a functional material accommodating article accommodating a functional material and an inhaler, wherein the functional material accommodating article may include a capsule accommodating a functional material, wherein the inhaler may include a housing including a first surface, a second surface that is opposite to the first surface, and a side surface between the first surface and the second surface, include an insertion space into which the functional material accommodating article is inserted from the first surface, and include an air inlet communicating with the insertion space, a sliding structure accommodated in the housing and movable in a longitudinal direction connecting the first surface to the second surface, and a needle accommodated in the housing and protruding from the second surface toward the first surface, wherein the sliding structure may include a sliding body on which the functional material accommodating article is mounted and which is movable in the longitudinal direction, wherein the sliding body may include a first sliding surface facing the first surface, a second sliding surface that is opposite to the first sliding surface, and a sliding side surface between the first sliding surface and the second sliding surface, a recessed part recessed from the first sliding surface, and an airflow hole extending from the sliding side surface to the recessed part, and wherein the air inlet and the airflow hole may communicate with each other.

According to an embodiment, an inhaler and an inhaling system including the inhaler may induce smooth discharge of a functional material.

According to an embodiment, an inhaler and an inhaling system including the inhaler may smoothly induce vortex behavior of air moving to a functional material accommodating article.

According to an embodiment, an inhaler and an inhaling system including the inhaler may implement tangential inflow of airflow.

According to an embodiment, an inhaler and an inhaling system including the inhaler may not require complex manufacturing facilities because forming a tangential airflow hole in a functional material accommodating article is not required.

According to an embodiment, an inhaler and an inhaling system including the inhaler may enhance aesthetics.

The effects of an inhaler and an inhaling system including the inhaler according to an embodiment are not limited to the above-mentioned effects, and other unmentioned effects may be clearly understood from the following description by one of ordinary skill in the art.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a perspective view of an inhaling system according to an embodiment;

FIG. 2A is a perspective view of an inhaler according to an embodiment;

FIG. 2B is a cross-sectional view taken along the line A-A of FIG. 2A;

FIG. 3A illustrates an inhaler excluding a housing, according to an embodiment;

FIG. 3B illustrates a sliding structure of the inhaler, according to an embodiment;

FIG. 4 illustrates another example of a sliding structure of an inhaler, according to an embodiment; and

FIG. 5 illustrates yet another example of a sliding structure of an inhaler, according to an embodiment.

### DETAILED DESCRIPTION

The terms used to describe the embodiments are selected from among common terms that are currently widely used, in consideration of their function in the embodiments. However, different terms may be used depending on an intention of one of ordinary skill in the art, a precedent, or the advent of new technology. Also, in particular cases, the terms are discretionally selected by the applicant of the disclosure, and the meaning of those terms will be described in detail in the corresponding part of the detailed description. Therefore, the terms used in the disclosure are not merely designations of the terms, but the terms are defined based on the meaning of the terms and content throughout the disclosure.

It will be understood that when a certain part “includes” a certain component, the part does not exclude another component but may further include another component, unless the context clearly dictates otherwise. Also, terms such as “unit,” “module,” etc., as used in the specification may refer to a part for processing at least one function or operation and may be implemented as hardware, software, or a combination of hardware and software.

As used herein, an expression such as “at least one of” that precedes listed components modifies not each of the listed components but all the components. For example, the expression “at least one of a, b, or c” should be construed as including a, b, c, a and b, a and c, b and c, or a, b, and c.

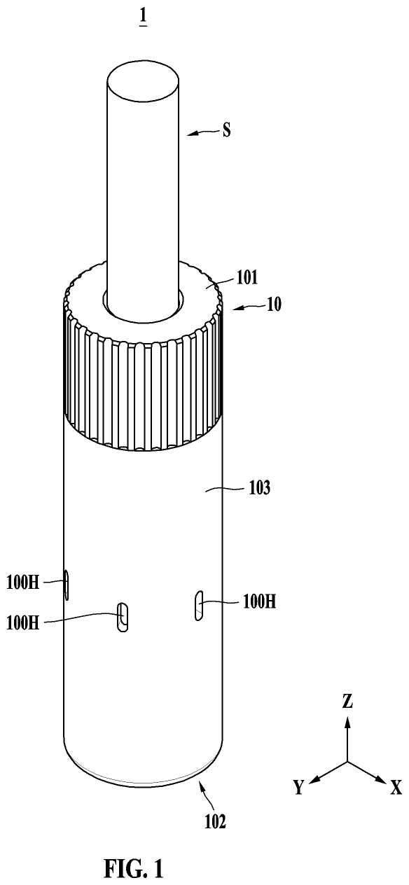

FIG. 1 is a perspective view of an inhaling system 1 according to an embodiment.

Referring to FIG. 1, the inhaling system 1 according to an embodiment may transfer a functional material to a user. For example, the inhaling system 1 according to an embodiment may include an inhaler that transfers nicotine to the lungs of the user in the form of an aerosol.

In an embodiment, the inhaling system 1 may include a functional material accommodating article S and an inhaler 10.

The functional material accommodating article S may be configured in the form of a stick. The stick may be configured in a cylindrical shape that may be inserted into the inhaler 10.

The functional material accommodating article S may include a plurality of segments in a longitudinal direction (e.g., +Z direction of FIG. 1). One of the plurality of segments may accommodate a capsule. The segment in which the capsule is accommodated may be configured as a cavity. For example, the capsule may be accommodated so as to be freely rotatable within the cavity, which may help a functional material in the capsule to easily escape from the capsule.

A front segment of the segment in which the capsule is accommodated may include a cellulose acetate material. The front segment may prevent leakage of functional material powder. A rear segment of the segment in which the capsule is accommodated may include a mouthpiece segment that is in contact with the mouth of the user. The segment in which the capsule is accommodated and the mouthpiece segment may be connected to each other to allow the flow a functional material and air.

The capsule may accommodate a functional material. For example, the functional material may include at least one of nicotine, theanine, caffeine, taurine, a pharmacological substance, or a mixture thereof. The functional material may have a form of fine granules or dry powder. In an example, an outer shell of the capsule may include a material that may be penetrated or cut by a needle 300. For example, the capsule may be filled with a functional material in an amount corresponding to an inhalation of 10 to 14 puffs by the user.

The functional material accommodating article S may be inserted into the inhaler 10. The capsule of the functional material accommodating article S accommodated in the inhaler 10 may be cut or crushed. In this case, the functional material may be inhaled by the user through the mouthpiece segment together with the air that exits from the capsule and enters the front segment of the functional material accommodating article S.

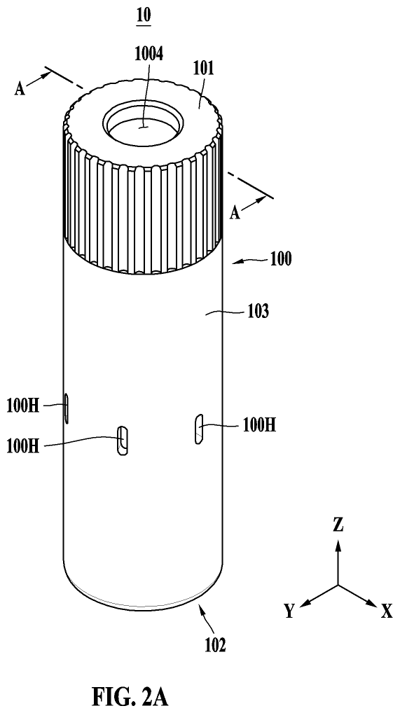

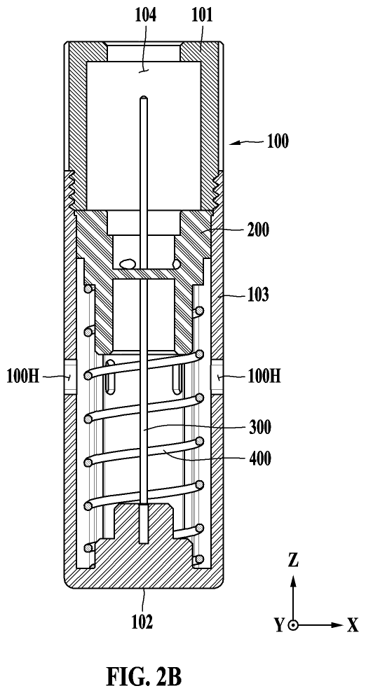

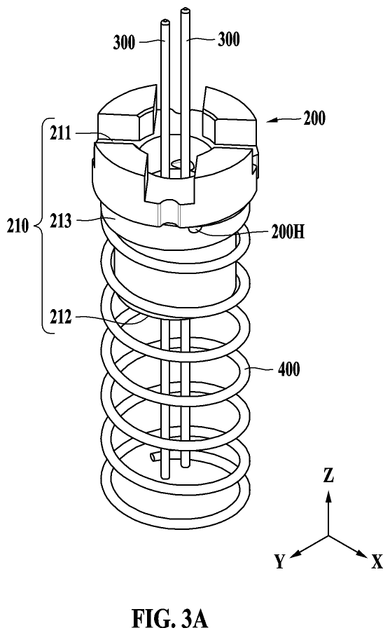

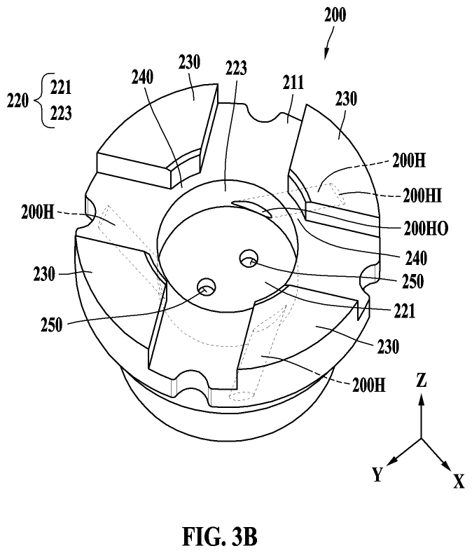

FIG. 2A is a perspective view of the inhaler 10 according to an embodiment, and FIG. 2B is a cross-sectional view taken along the line A-A of FIG. 2A. FIG. 3A illustrates the inhaler 10 excluding a housing 100, according to an embodiment, and FIG. 3B illustrates a sliding structure 200 of the inhaler 10, according to an embodiment.

Referring to FIGS. 2A and 2B, the inhaler 10 according to an embodiment may include the housing 100, the sliding structure 200, the needle 300, and an elastic body 400.

In an embodiment, the housing 100 may include a first surface 101, a second surface 102 that is opposite to the first surface 101, and a side surface 103 between the first surface 101 and the second surface 102.

An insertion space 104 into which a functional material accommodating article S is inserted may be formed on the first surface 101 of the housing 100. An air inlet 100H communicating with the insertion space 104 may be formed in the housing 100. For example, the air inlet 100H may be provided on the side surface 103 of the housing 100. A plurality of air inlets 100H may be provided, and the plurality of air inlets 100H may be arranged at equal intervals along the perimeter of the side surface 103.

In an embodiment, the sliding structure 200 may be accommodated in the housing 100. The sliding structure 200 may move in a longitudinal direction (e.g., +Z direction in FIG. 2A) extending the first surface 101 and the second surface 102.

The elastic body 400 may be accommodated in the housing 100. The elastic body 400 may be disposed between the sliding structure 200 and the second surface 102 of the housing 100 and may apply elastic force to the sliding structure 200 toward the first surface (e.g., toward the +Z direction in FIG. 2B). For example, the elastic body 400 may include a coil spring.

In an embodiment, the needle 300 may be accommodated in the housing 100. One end of the needle 300 may be fixed to the second surface 102 of the housing 100, and the other end (tip) of the needle 300 may protrude toward the insertion space 104 of the housing 100.

For example, the needle 300 may be provided in plurality. In particular, referring to FIG. 3A, two needles 300 may be arranged parallel in a longitudinal direction (e.g., Z direction). When the two needles 300 are inserted into the functional material accommodating article S, the functional material accommodating article S may be prevented from rotating in the insertion space 104.

The needle 300 may penetrate the sliding structure 200. In particular, referring to FIG. 3B, the sliding structure 200 may be provided with a needle hole 250 penetrating the sliding structure 200 in the longitudinal direction (e.g., Z direction), and the needle 300 may extend through the needle hole 250 by penetrating the sliding structure 200.

Referring to FIGS. 3A and 3B, the sliding structure 200 may include a sliding body 210, a recessed part 220, a protruding part 230, a seating part 240, and an airflow hole 200H.

The sliding body 210 may include a first sliding surface 211 facing the first surface 101 of the housing 100, a second sliding surface 212 that is opposite to the first sliding surface 211, and a sliding side surface 213 between the first sliding surface 211 and the second sliding surface 212.

The sliding side surface 213 may be provided in a stepped shape. The upper sliding side surface 213 adjacent to the first sliding surface 211 may have a wider width than the lower sliding side surface 213 adjacent to the second sliding surface 212. Referring to FIG. 2B, the upper sliding side surface 213 may be in contact with an inner wall of the housing 100, and the lower sliding side surface 213 may be spaced apart from the inner wall of the housing 100.

The airflow hole 200H may be formed in the lower sliding side surface 213. Since the airflow hole 200H is positioned in the lower sliding side surface 213, external air passing through the air inlet 100H of the housing 100 may easily enter the airflow hole 200H without being interrupted by the inner wall of the housing 100.

The recessed part 220 may have a structure that is recessed from the first sliding surface 211. The recessed part 220 may include a recessed base 221 that is recessed from the first sliding surface 211 to the second sliding surface 212 and a recessed side surface 223 that extends from the recessed base 221 to the first sliding surface 211. The upper part of the recessed part 220 (e.g., the upper part in the +Z direction in FIG. 3B) may be an open space.

The needle hole 250 may be formed by penetrating from the recessed base 221 to the second sliding surface 212.

The airflow hole 200H may be formed by penetrating from the sliding side surface 213 to the recessed side surface 223.

The protruding part 230 may protrude from the first sliding surface 211 toward the first surface 101 of the housing 100. The protruding part 230 may be disposed on the outside of the recessed part 220.

For example, the protruding part 230 may be provided in plurality. A plurality of protruding parts 230 may be arranged to be spaced apart from the recessed part 220 at equal distances outwardly (e.g., in the +X direction or +Y direction in FIG. 3B). The functional material accommodating article S may be introduced into a region formed by being surrounded by the protruding parts 230. In this case, the functional material accommodating article S may be restricted from moving outward (e.g., in the +X direction or +Y direction in FIG. 3B) by the protruding parts 230.

The seating part 240 may be disposed between the protruding part 230 and the recessed part 220. The seating part 240 may be defined as a region of the first sliding surface 211 between the protruding part 230 and the recessed part 220.

The diameter of the region formed by being surrounded by the protruding parts 230 may be greater than the diameter of an upper region of the recessed part 220 on the same plane as the first sliding surface 211. In addition, the diameter of the region formed by being surrounded by the protruding parts 230 may be greater than the diameter (e.g., a diameter in an XY plane) of the front end of the functional material accommodating article S. The diameter of the upper region of the recessed part 220 may be less than the diameter of the front end of the functional material accommodating article S. By this relationship, the front end of the functional material accommodating article S may be seated on the seating part 240. Furthermore, a portion of the front end of the functional material accommodating article S may be exposed to the recessed part 220, the front end of the functional material accommodating article S and an outlet 200HO of the airflow hole 200H may be spaced apart from each other, and air exiting from the outlet 200HO of the airflow hole 200H may form a vortex in the recessed part 220 and then enter the functional material accommodating article S. The formation of a vortex is described below.

In an embodiment, a virtual line connecting an inlet 200HI of the airflow hole 200H formed in the sliding side surface 213 to the outlet 200HO of the airflow hole 200H formed in the recessed side surface 223 may be biased from the center (e.g., the center of the recessed base 221) of the sliding body 210. When the airflow hole 200H is formed diagonally (formed so as to face a tangential direction) rather than toward the center of the recessed base 221, a vortex may be formed in the recessed part 220 as the air entering the airflow hole 200H exits through the outlet 200HO of the airflow hole 200H. The vortex formed in this way may enter the functional material accommodating article S and induce rotation and vibration of a capsule. The functional material accommodated in the capsule may easily escape from the capsule by the rotation and vibration of the capsule.

A plurality of airflow holes 200H may be arranged at equal intervals around the perimeter of the recessed part 220. Each airflow hole 200H may be arranged to face in a diagonal direction (tangential direction).

In an embodiment, the area of the outlet 200HO of the airflow hole 200H may be less than the area of the inlet 200HI of the airflow hole 200H. Air entering the inlet 200HI of the airflow hole 200H may be accelerated due to the area gradually narrowing toward the outlet 200HO of the airflow hole 200H. The flow velocity of the air exiting from the outlet 200HO of the airflow hole 200H may be faster than the flow velocity of the air entering the inlet 200HI of the airflow hole 200H. A fast flow velocity of air may facilitate vortex formation.

For example, the airflow hole 200H may extend in a straight line. The airflow hole 200H may be formed along a straight virtual line connecting the inlet 200HI to the outlet 200HO of the airflow hole 200H. The airflow hole 200H in a straight shape may increase the convenience of processing the airflow hole 200H.

In another example, the airflow hole 200H may extend in a curved line. The airflow hole 200H in a curved shape may bend the flow of air entering the recessed part 220, so a vortex may be formed more smoothly along the recessed side surface 223.

Hereinafter, the operation of the inhaling system 1 according to an embodiment is described with reference to FIGS. 1, 2A, 2B 3A and 3B.

The functional material accommodating article S may be inserted into the insertion space 104 of the housing 100 of the inhaler 10, and the front end of the functional material accommodating article S may be seated on the seating part 240 of the sliding body 210. In this case, the needle 300 may be inserted into at least a portion of the functional material accommodating article S.

In this state, when the functional material accommodating article S is continuously inserted into the insertion space 104, the sliding body 210 together with the functional material accommodating article S may move toward the second surface 102 of the housing 100 (e.g., toward the −Z direction). In this case, the capsule in the functional material accommodating article S may be crushed or cut by the needle 300, and a functional material accommodated in the capsule may be exposed the outside of the capsule.

When the force applied to the functional material accommodating article S is removed, the sliding structure 200 may return to the original position of the sliding structure 200 by the elastic body 400, and the functional material accommodating article S may also move together with the sliding structure 200. When the sliding structure 200 completes returning to the original position of the sliding structure 200, the functional material accommodating article S may stay inserted into the housing 100 while seated on the sliding structure 200. In this state, a user may inhale a functional material by holding a mouthpiece segment of the functional material accommodating article S in the mouth of the user.

When the user starts inhaling, negative pressure is formed in the recessed part 220 of the sliding structure 200 through the mouthpiece segment. Due to the negative pressure of the recessed part 220, air outside the housing 100 may be introduced into the recessed part 220 through the air inlet 100H and the airflow hole 200H. In this case, due to the structure and arrangement of the airflow hole 200H, air may be introduced into the recessed part 220 in a diagonal direction (tangential direction), and a vortex air flow may be formed in the recessed part 220. This vortex air flow may enter the functional material accommodating article S and cause rotation and vibration of the capsule. A functional material that smoothly exits from the capsule by the rotation and vibration of the capsule may be inhaled by the user through the mouthpiece segment.

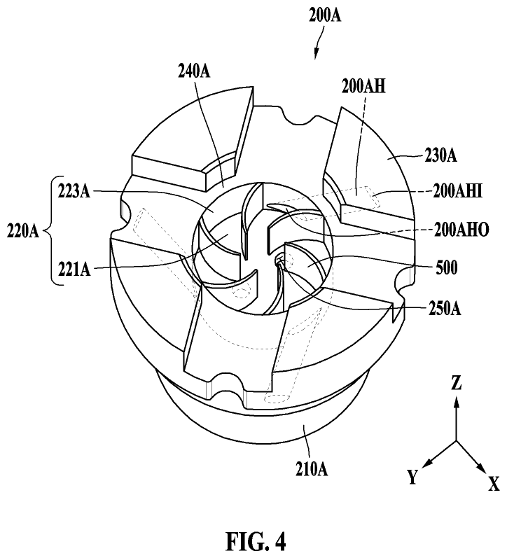

FIG. 4 illustrates another example of a sliding structure of an inhaler, according to an embodiment. Referring to FIG. 4, among components of a sliding structure 200A, descriptions of components identical or similar to the components of the sliding structure 200 of FIGS. 2A to 3B are omitted for simplicity.

Referring to FIG. 4, the sliding structure 200A may include a sliding body 210A, a recessed part 220A, a protruding part 230A, a seating part 240A, an airflow hole 200AH, and an impeller 500.

In an embodiment, the impeller 500 may be formed in a recessed base 221A. The impeller 500 may extend from a recessed side surface 223A to the center of the recessed base 221A. The impeller 500 may be disposed at a position that does not interfere with a needle hole 250A.

The impeller 500 may be provided in plurality, and an outlet 200AHO of the airflow hole 200AH may be positioned between impellers 500 that are adjacent to one another.

A virtual line connecting an inlet 200AHI of the airflow hole 200AH formed in a sliding side surface 213A to an outlet 200AHO of the airflow hole 200AH formed in a recessed side surface 223A may be biased from the center (e.g., the center of the recessed base 221A) of the sliding body 210A. When the airflow hole 200AH is formed in a diagonal direction (formed so as to face a tangential direction) rather than toward the center of the recessed base 221A, a vortex may be formed in the recessed part 220A as the air entering the airflow hole 200AH exits through the outlet 200AHO of the airflow hole 200AH. In this case, the air that exits through the outlet 200AHO of the airflow hole 200AH may form a vortex more smoothly by passing through the impeller 500 once more.

The impeller 500 may have a curved shape. In more detail, the impeller 500 may have a blade or plate shape that curves from the recessed side surface 223A toward the recessed base 221A.

In an embodiment, a spacing between the impellers 500 that are adjacent to one another may narrow from the recessed side surface 223A toward the center of the recessed base 221A. The air that exits from the outlet 200AHO of the airflow hole 200AH may be accelerated due to an area that gradually narrows as the air passes through the impeller 500. The flow velocity of the air exiting from the impeller 500 may be faster than the flow velocity of the air exiting from the outlet 200AHO of the airflow hole 200AH. A fast flow velocity of air may facilitate vortex formation.

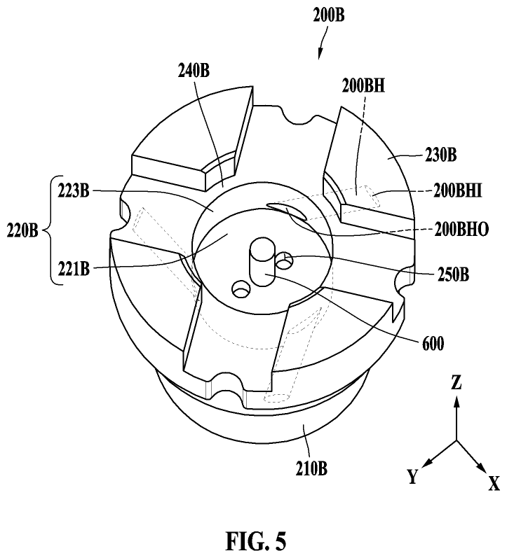

FIG. 5 illustrates yet another example of a sliding structure of an inhaler, according to an embodiment. Referring to FIG. 5, among components of a sliding structure 200B, descriptions of components identical or similar to the components of the sliding structure 200 of FIGS. 2A to 3B are omitted for simplicity.

Referring to FIG. 5, the sliding structure 200B may include a sliding body 210B, a recessed part 220B, a protruding part 230B, a seating part 240B, an airflow hole 200BH, and an airflow guide 600.

In an embodiment, the airflow guide 600 may be a structure that protrudes upward (toward the +Z direction) from the center of a recessed base 221B. The airflow guide 600 may guide a vortex formed in the recessed part 220B to the functional material accommodating article S.

The air exiting from an outlet 200BHO of the airflow hole 200BH may gather at the center of the recessed part 220B and hit the airflow guide 600. The air that hits the airflow guide 600 may be smoothly guided upward along an outer surface of the airflow guide 600. The air that escapes from the airflow guide 600 may enter the functional material accommodating article S.

According to an embodiment, the inhaler 10 and the inhaling system 1 including the inhaler 10 may induce smooth discharge of a functional material and smoothly induce vortex behavior of air moving to a functional material accommodating article. In addition, in implementing the tangential inflow of airflow, there is no need to form a tangential airflow hole in the functional material accommodating article S, so there is an advantage of not having to have complex manufacturing equipment.

According to an embodiment, the functional material accommodating article S accommodating a functional material may be inserted into the inhaler 10. The inhaler 10 may include the housing 100 including the first surface 101, the second surface 102 that is opposite to the first surface 101, and the side surface 103 between the first surface 101 and the second surface 102 and comprising the insertion space 104 into which the functional material accommodating article S is inserted from the first surface 101, the sliding structure 200 accommodated in the housing 100 and movable in a longitudinal direction connecting the first surface 101 to the second surface 102, and the needle 300 accommodated in the housing 100 and protruding from the second surface 102 toward the first surface 101, wherein the air inlet 100H communicating with the insertion space 104 may be formed in the housing 100, and air outside the housing 100 moves through the air inlet 100H, passes through the sliding structure 200, and reaches the functional material accommodating article S.

In an embodiment, the sliding structure 200 may include the sliding body 210 on which the functional material accommodating article S is mounted and which is movable in the longitudinal direction and the airflow hole 200H formed in the sliding body 210, and the sliding body 210 may include the first sliding surface 211 facing the first surface 101, the second sliding surface 212 that is opposite to the first sliding surface 211, and the sliding side surface 213 between the first sliding surface 211 and the second sliding surface 212.

The sliding structure 200 may further include the recessed part 220 recessed from the first sliding surface 211, the protruding part 230 protruding from the first sliding surface 211 and disposed outside the recessed part 220, and the seating part 240 between the recessed part 220 and the protruding part 230, the recessed part 220 may include the recessed base 221 and the recessed side surface 223 extending from the recessed base 221 to the first sliding surface 211, and the airflow hole 200H may be formed to penetrate from the sliding side surface 213 to the recessed side surface 223.

The virtual line connecting the inlet 200HI of the airflow hole 200H positioned in the sliding side surface 213 to the outlet 200HO of the airflow hole 200H positioned in the recessed side surface 223 may be biased from the center of the sliding body 210.

In an embodiment, the area of the outlet 200HO of the airflow hole 200H may be less than the area of the inlet 200HI of the airflow hole 200H.

In an embodiment, the airflow hole 200H may extend in a straight line.

In an embodiment, the airflow hole 200H may extend in a curved line.

The sliding structure 200A according to an embodiment may further include the impeller 500 formed in the recessed base 221A.

The impeller 500 may extend from the recessed side surface 223A to the center of the recessed base 221A.

The impeller 500 may include impellers, and the outlet 200AHO of the airflow hole 200AH may be positioned between impellers 500 that are adjacent to one another.

The impeller 500 may have a curved shape.

In an embodiment, a spacing between neighboring impellers 500 may narrow from the recessed side surface 223A toward the center of the recessed base 221A.

The sliding structure 200B according to an embodiment may further include the airflow guide 600 protruding from the center of the recessed base 221B toward the first surface 101.

The inhaling system 1 may include the functional material accommodating article S accommodating the functional material and the inhaler 10 into which the functional material accommodating article S is inserted, wherein the functional material accommodating article S may include a capsule accommodating the functional material, wherein the inhaler 10 may include the housing 100 including the first surface 101, the second surface 102 that is opposite to the first surface 101, and the side surface 103 between the first surface 101 and the second surface 102, including the insertion space 104 into which the functional material accommodating article S is inserted from the first surface 101, and including the air inlet 100H communicating with the insertion space 104, the sliding structure 200 accommodated in the housing 100 and movable in a longitudinal direction connecting the first surface 101 to the second surface 102, and the needle 300 accommodated in the housing 100 and protruding from the second surface 102 toward the first surface 101, wherein the sliding structure 200 may include the sliding body 210 on which the functional material accommodating article S is mounted and which is movable in the longitudinal direction, wherein the sliding body 210 may include the first sliding surface 211 facing the first surface 101, the second sliding surface 212 that is opposite to the first sliding surface 211, and the sliding side surface 213 between the first sliding surface 211 and the second sliding surface 212, the recessed part 220 recessed from the first sliding surface 211, and the airflow hole 200H extending from the sliding side surface 213 to the recessed part 220, and wherein the air inlet 100H and the airflow hole 200H may communicate with each other.

The virtual line connecting the inlet 200HI of the airflow hole 200H positioned in the sliding side surface 213 to the outlet 200HO of the airflow hole 200H positioned in the recessed part 220 may be biased from the center of the sliding body 210.

The descriptions of the above-described embodiments are only examples, and it will be understood by one of ordinary skill in the art that various changes and equivalents may be made thereto. Therefore, the scope of the disclosure should be defined by the appended claims, and all differences within the scope equivalent to those described in the claims will be construed as being included in the scope of protection defined by the claims.

What is claimed is:

1. An inhaler into which a functional material accommodating article accommodating a functional material is inserted, the inhaler comprising:
a housing comprising a first surface, a second surface that is opposite to the first surface, and a side surface between the first surface and the second surface and comprising an insertion space into which the functional material accommodating article is inserted from the first surface;
a sliding structure accommodated in the housing and movable in a longitudinal direction connecting the first surface to the second surface; and
a needle accommodated in the housing and protruding from the second surface toward the first surface,
wherein an air inlet communicating with the insertion space is formed in the housing, and air outside the housing moves through the air inlet, passes through the sliding structure, and reaches the functional material accommodating article.

2. The inhaler of claim 1, wherein
the sliding structure comprises:
a sliding body on which the functional material accommodating article is mounted and which is movable in the longitudinal direction; and
an airflow hole formed in the sliding body, and
the sliding body comprises a first sliding surface facing the first surface, a second sliding surface that is opposite to the first sliding surface, and a sliding side surface between the first sliding surface and the second sliding surface.

3. The inhaler of claim 2, wherein
the sliding structure further comprises:
a recess part recessed from the first sliding surface;
a protruding part protruding from the first sliding surface and disposed outside the recessed part; and
a seating part between the recessed part and the protruding part,
the recessed part comprises a recessed base and a recessed side surface extending from the recessed base to the first sliding surface, and
the airflow hole is formed to penetrate from the sliding side surface to the recessed side surface.

4. The inhaler of claim 3, wherein a virtual line connecting an inlet of the airflow hole positioned in the sliding side surface to an outlet of the airflow hole positioned in the recessed side surface is biased from a center of the sliding body.

5. The inhaler of claim 4, wherein an area of the outlet of the airflow hole is less than an area of the inlet of the airflow hole.

6. The inhaler of claim 4, wherein the airflow hole extends in a straight line.

7. The inhaler of claim 4, wherein the airflow hole extends in a curved line.

8. The inhaler of claim 4, further comprising:
an impeller formed on the recessed base.

9. The inhaler of claim 8, wherein the impeller extends from the recessed side surface to a center of the recessed base.

10. The inhaler of claim 9, wherein the impeller comprises impellers, and the outlet of the airflow hole is positioned between the impellers that are adjacent to one another.

11. The inhaler of claim 10, wherein the impeller has a curved shape.

12. The inhaler of claim 10, wherein a spacing between the impellers that are adjacent to one another narrows from the recessed side surface toward the center of the recessed base.

13. The inhaler of claim 4, further comprising:
an airflow guide protruding from a center of the recessed base toward the first surface.

14. An inhaling system comprising:
a functional material accommodating article accommodating a functional material; and
an inhaler into which the functional material accommodating article is inserted,
wherein the functional material accommodating article comprises a capsule accommodating a functional material,
wherein the inhaler comprises:
a housing comprising a first surface, a second surface that is opposite to the first surface, and a side surface between the first surface and the second surface, comprising an insertion space into which the functional material accommodating article is inserted from the first surface, and comprising an air inlet communicating with the insertion space;
a sliding structure accommodated in the housing and movable in a longitudinal direction connecting the first surface to the second surface; and
a needle accommodated in the housing and protruding from the second surface toward the first surface,
wherein the sliding structure comprises:
a sliding body on which the functional material accommodating article is mounted and which is movable in the longitudinal direction, wherein the sliding body comprises a first sliding surface facing the first surface, a second sliding surface that is opposite to the first sliding surface, and a sliding side surface between the first sliding surface and the second sliding surface;
a recessed part recessed from the first sliding surface; and
an airflow hole extending from the sliding side surface to the recessed part, and
wherein the air inlet and the airflow hole communicate with each other.

15. The inhaling system of claim 14, wherein a virtual line connecting an inlet of the airflow hole positioned in the sliding side surface to an outlet of the airflow hole positioned in the recessed part is biased from a center of the sliding body.
